(19) United States
(12) Patent Application Publication
(10) Pub. No.: US 2013/0103103 A1
(43) Pub. Date: Apr. 25, 2013

(54) SURGICAL SYSTEM METHODS FOR SPINAL ACCESS

(71) Applicants: David A. Mire, Cordova, TN (US); Paula A. Nichter, Memphis, TN (US); Kelli N. Sebastian, Arlington, TN (US); Bryant Lee, Memphis, TN (US)

(72) Inventors: David A. Mire, Cordova, TN (US); Paula A. Nichter, Memphis, TN (US); Kelli N. Sebastian, Arlington, TN (US); Bryant Lee, Memphis, TN (US)

(73) Assignee: WARSAW ORTHOPEDIC, INC, Warsaw, IN (US)

(21) Appl. No.: 13/280,010

(22) Filed: Oct. 24, 2011

(51) Int. Cl. A61B 17/56; A61B 1/32

(52) U.S. Cl. 606/86 A; 600/235

(57) ABSTRACT

A dilator includes a first portion and a second portion movable between a first position and a second position. The first portion includes a first wall that defines a first cross section and a first cavity. The second portion includes a second wall that defines a second cross section, which is less than the first cross section, and a second cavity. The second portion dilates and/or dissects tissue adjacent a surgical site. In the first position, the second cavity provides direct visualization of the surgical site and the first cavity supports an instrument. In the second position, the second cavity is expandable such that the instrument is movable along the longitudinal axis in the second cavity. Methods of use are disclosed.

### TECHNICAL FIELD

The present disclosure generally relates to medical devices for the treatment of musculoskeletal disorders, and more particularly to a surgical system and method for accessing a spine to facilitate treatment.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, discectomy, laminectomy and implantable prosthetics. Percutaneous and other minimally invasive surgical procedures can be employed to minimize disruption and trauma to a patient's body to reduce recovery time and post-operative pain. Surgical instruments are used in such minimally invasive procedures to increase the workspace of a minimally invasive surgical incision and adjacent areas used to access a surgical site. This disclosure describes an improvement over these prior art technologies.

### SUMMARY

Accordingly, a surgical system and method are provided for accessing a spine to facilitate treatment thereof. It is contemplated that the surgical system and method may be employed for dilating and/or dissecting tissue to access a surgical site, which may be performed under direct visualization. It is further contemplated that the surgical system and method may be employed for lateral access to a spine through the psoas muscle of a patient.

In one embodiment, in accordance with the principles of the present disclosure, a system for treating a spine is provided. The system includes a dilator defining a longitudinal axis and including a first portion and a second portion extending from the first portion. The dilator is movable between a first position and a second position. The first portion of the dilator includes a first wall configured to define a first cross section dimension of the first portion and a first cavity extending along the longitudinal axis. The second portion includes a second wall configured to define a second cross section dimension of the second portion and a second cavity extending along the longitudinal axis. The second cross section dimension is less than the first cross section dimension. The second portion further defines an outer surface configured to dilate and/or dissect tissue adjacent a surgical site. In the first position, the second cavity is configured to provide direct visualization of the surgical site and the first cavity is configured to support a surgical instrument. In the second position, the second cavity is expandable to a configuration such that the surgical instrument is movable along the longitudinal axis in the second cavity.

In one embodiment, the system includes a speculum. The speculum includes a first arm and a second arm connected to the first arm. The second arm is movable relative to the first arm. The first arm and the second arm define a proximal portion of the speculum. The proximal portion includes a first of the first arm and a first wall of the second arm. The first wall of the first arm and the first wall of the second arm are configured to define a cross section dimension of the proximal portion and a proximal cavity extending along a longitudinal axis thereof. The first arm and the second arm further define a distal portion of the speculum. The distal portion includes a second wall of the first arm and a second wall of the second arm. The second wall of the first arm and the second wall of the second arm are configured to define a second cross section dimension of the distal portion and a distal cavity extending along the longitudinal axis. The second cross section dimension is less than the first cross section dimension. The distal portion further defines an outer surface configured to dilate and/or dissect tissue adjacent a surgical site. In a first position, the distal cavity is disposable in a non-expanded configuration to provide direct visualization of the surgical site and the proximal cavity is configured to support a surgical instrument. In a second position, the distal cavity is expandable such that the second wall of the first arm and the second wall of the second arm are spaced apart and the surgical instrument is movable along the longitudinal axis in the distal cavity.

In one embodiment, a method for providing surgical access to a spine is provided, which includes the steps of providing a dilator including a first portion and a second portion extending from the first portion. The first portion includes a first wall configured to define a first cross section dimension of the first portion and a first cavity. The second portion includes a second wall configured to define a second cross section dimension of the second portion and a second cavity. The second cross section dimension is less than the first cross section dimension. The dilator is disposed for engagement with psoas tissue of a patient such that the second cavity is non-expanded and oriented to provide direct visualization of a surgical site, and the first cavity is configured to support a surgical instrument. The second portion is advanced through the psoas tissue to the surgical site. The second cavity is expanded to a configuration such that the surgical instrument is movable along the longitudinal axis in the second cavity. The surgical instrument is advanced to the surgical site to provide access thereto.

### BRIEF DESCRIPTION OF THE DRAWINGS

The present disclosure will become more readily apparent from the specific description accompanied by the following drawings, in which:

FIG. 1 is a perspective view of one particular embodiment of a dilator of a system in accordance with the principles of the present disclosure;

FIG. 2 is a perspective view of the dilator shown in FIG. 1 with a retractor of the system;

FIG. 3 is a side view of the system shown in FIG. 2 accessing a surgical target site;

FIG. 4 is a perspective view of the system shown in FIG. 2;

FIG. 5 is a perspective view of the retractor shown in FIG. 2;

FIG. 6 is a perspective view of one embodiment of a system in accordance with the principles of the present disclosure;

FIG. 7 is a perspective view of the system shown in FIG. 6 with a tip removed.;

FIG. 8 is a perspective view of one embodiment of a dilator of a system in accordance with the principles of the present disclosure;

FIG. 9 is a perspective view of the dilator shown in FIG. 8 with parts separated;

FIG. 10 is a side view of the system shown in FIG. 8 with a retractor;

FIG. 11 is a perspective view of the system shown in FIG. 10;

FIG. 12 is a perspective view of one embodiment of a dilator in accordance with the principles of the present disclosure;

FIG. 13 is a side view of the dilator shown in FIG. 12;

FIG. 14 is a side view, in part cutaway, of the dilator shown in FIG. 12;

FIG. 15 is a side view of the dilator shown in FIG. 12 with parts separated;

FIG. 16 is a side view of the dilator shown in FIG. 12;

FIG. 17 is a perspective view of one embodiment of a dilator in accordance with the principles of the present disclosure;

FIG. 18 is a cutaway perspective view of the system shown in FIG. 17;

FIG. 19 is a perspective view of one embodiment of a dilator in accordance with the principles of the present disclosure; and

FIG. 20 is a cutaway perspective view of the system shown in FIG. 19.

Like reference numerals indicate similar parts throughout the figures.

### DETAILED DESCRIPTION

The exemplary embodiments of the surgical system and related methods of use disclosed are discussed in terms of medical devices for the treatment of musculoskeletal disorders and more particularly, in terms of a surgical system for accessing a spine to facilitate treatment thereof and a method for treating a spine. In one embodiment, the surgical system and methods presently disclosed facilitate lateral access to a spine through a psoas muscle in a configuration and orientation to avoid surgical and post-surgical complications. It is envisioned that the surgical system and methods of use disclosed can be employed to perform a discectomy through a minimally invasive or percutaneous technique. It is further envisioned that the disclosed system and methods can be used in connection with and/or to supplement an instrumented minimally invasive or percutaneous interbody fusion. In one embodiment, the surgical system and methods of use disclosed are designed to avoid undesirable engagement or interference with body structures. One or all of the system components may be reusable or disposable. The surgical system may be configured as a kit with multiple sized and configured components.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

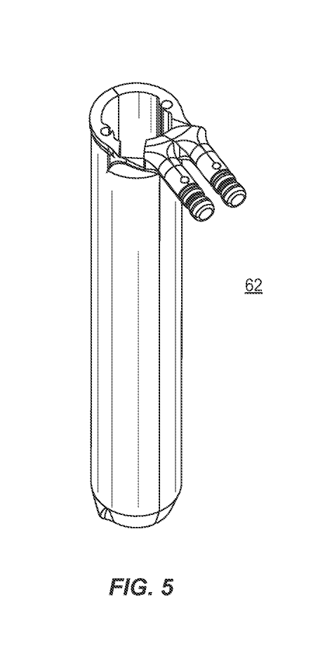

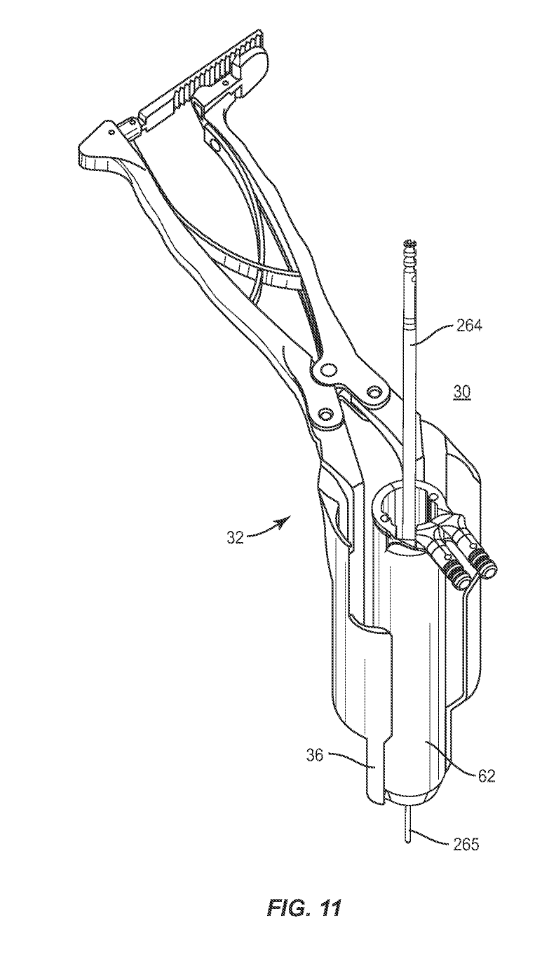

The following discussion includes a description of a surgical system and related methods of employing the surgical system in accordance with the principles of the present disclosure. Alternate embodiments are also disclosed. Reference will now be made in detail to the exemplary embodiments of the present disclosure, which are illustrated in the accompanying figures. Turning now to FIGS. 11-5, there is illustrated components of a surgical system 30 for accessing a spine to facilitate treatment thereof in accordance with the principles of the present disclosure.

The components of system 30 can be fabricated from biologically acceptable materials suitable for medical applications, including metals, synthetic polymers, ceramics and/or their composites, depending on the particular application and/or preference of a medical practitioner. For example, the components of system 30, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomer composites, rigid polymers including polyphenylene, polyamide, polyimide, polyetherimide, polyethylene, epoxy, composites of PEEK and calcium based ceramics, and composites of PEEK with resorbable polymers. Various components of system 30 may have material composites, including the above materials, to achieve various desired characteristics such as strength, rigidity, elasticity, compliance, biomechanical performance, durability and radiolucency or imaging preference. The components of system 30, individually or collectively, may also he fabricated from a heterogeneous material such as a combination of two or more of the above-described materials. The components of system 30 may be monolithically formed, integrally connected or include fastening elements and/or instruments, as described herein.

System 30 is employed, for example, with a minimally invasive procedure, including percutaneous techniques, to provide access to a spine to facilitate treatment. In one embodiment, the components of system 30 are configured to provide lateral access to the spine through the psoas muscle to create a void between muscle fibers by using direct visualization to dissect through the psoas muscle and other tissue.

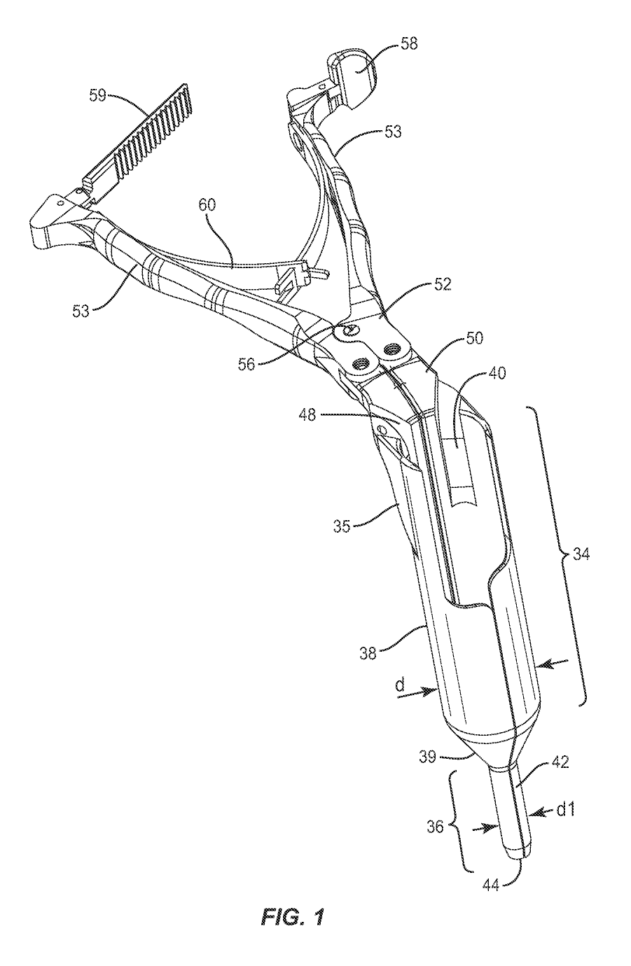

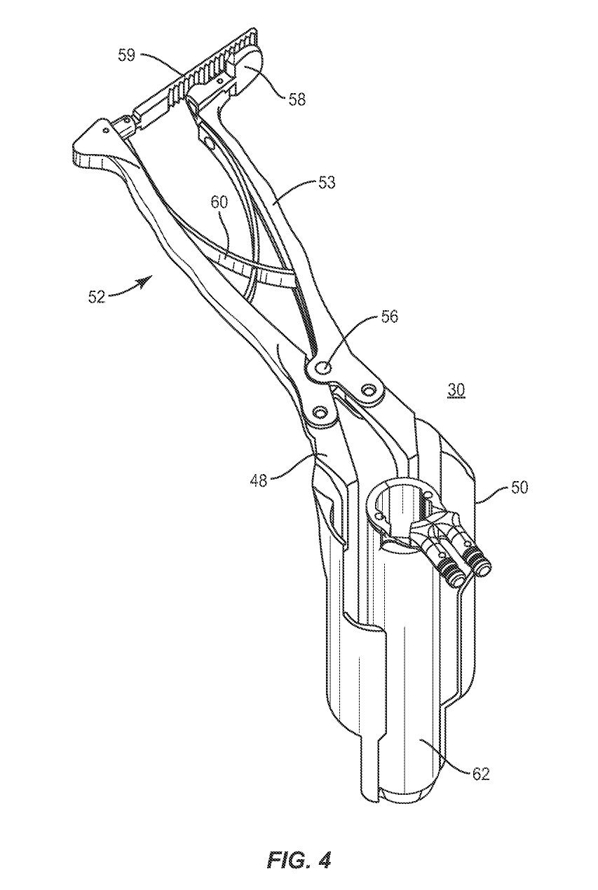

System 30 includes a dilator 32 that defines a longitudinal axis a and includes a first portion, such as, for example, proximal portion 34 and a second portion, such as, for example, distal portion 36 extending from proximal portion 34. Dilator 32 is movable between a first configuration, such as, for example, a non-expanded configuration, as shown in FIGS. 1-3, and a second configuration, such as, for example, an expanded configuration, as shown in FIG. 4. It is contemplated that system 30 may include one or a plurality of dilators 32 and/or employ other surgical instruments such as, for example, a penfield, forceps and/or other dilators to gradually separate muscle and/or tissue to create a portal including a passageway to a surgical target site adjacent the spine. it is further contemplated that dilator 32 may be configured as an in-situ guidance instrument and system 30 may include an endoscope. Dilator 32 is configured to be inserted via lateral access into an incision over a surgical site to provide direct visualization of the surgical site and/or position a surgical instrument within the surgical site.

Proximal portion 34 of dilator 32 includes a first wall, such as, for example, a bifurcated cylinder 38. Bifurcated cylinder 38 defines a first cavity, such as, for example, a proximal cavity 40 extending along longitudinal axis a defined by dilator 32. Cylinder 38 has a first cross section dimension, such as, for example, a diameter d of proximal portion 34. It is contemplated that the first cross section dimension may alternatively include thickness, height, length or width depending on the geometry of the first wall. Dilator 32 may include a proximal portion 34 having an adjustable axial length such that dilator 32 is adjustable to conform to an outer surface of the patient.

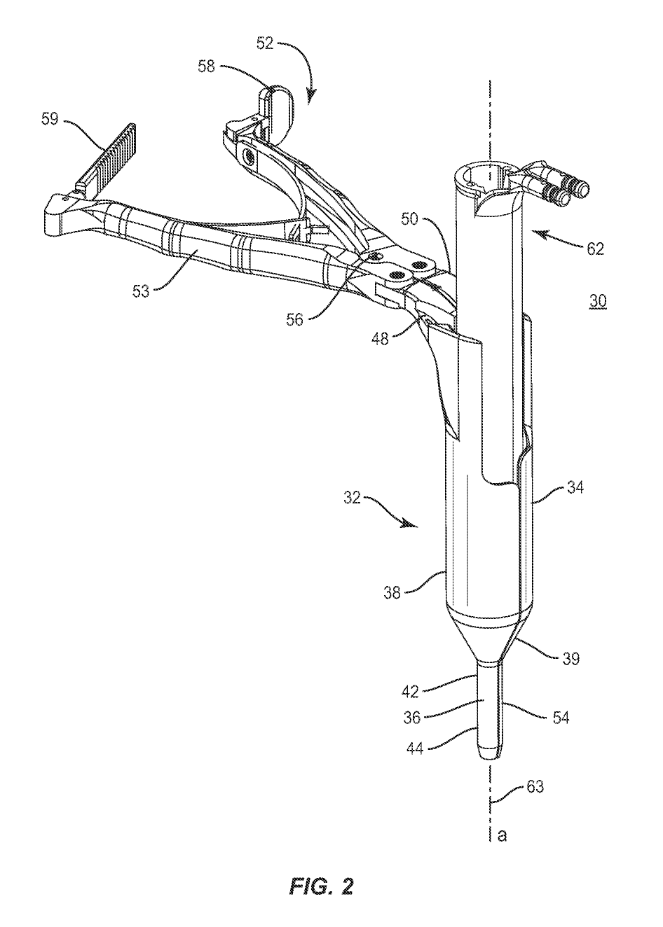

In the non-expanded configuration, proximal portion 34 is configured to support a surgical instrument within proximal cavity 40 and facilitate direct visualization of a surgical site with distal portion 36, as will be described. In the expanded configuration, proximal portion 34 is expanded from the non-expanded configuration, as described below, to facilitate passage of the surgical instrument therethrough. Proximal portion 34 includes an open proximal end 35 such that a surgical instrument supported in proximal cavity 40 may extend above proximal end 35, thereby allowing proximal portion 34 to support a surgical instrument having a length that is greater than the length of proximal portion 34, as shown in FIG. 2, for example. It is envisioned that proximal end 35 may be partially or completely closed such that a surgical instrument supported by proximal portion 34 may be positioned entirely within proximal cavity 40.

Bifurcated cylinder 38 has a smooth or even inner and outer surface. It is envisioned that all or only a portion of the inner and outer surfaces of bifurcated cylinder 38 may have alternate surface configurations, such as, for example, rough, threaded for connection with other instruments, arcuate, undulating, porous, semi-porous, dimpled, polished and/or textured according to the requirements of a particular application. It is envisioned that proximal portion 34 and/or proximal cavity 40 may have alternate cross section configurations, such as, for example, oval, oblong, triangular, rectangular, square, polygonal, irregular, uniform, non-uniform, variable and/or tapered. It is further contemplated that proximal portion 34 may include fastening elements such as anchors, detents and/or openings for connection to surgical instruments.

Distal portion 36 of dilator 32 includes a second wall, such as, for example, a bifurcated cylinder 42. Bifurcated cylinder 42 is configured to define a distal cavity 44 extending along longitudinal axis a. Cylinder 42 has a second cross section dimension, such as, for example, a diameter d1 of distal portion 36. Diameter d1 of cylinder 42 is less than diameter d of cylinder 38. It is contemplated that the second cross section dimension may alternatively include thickness, height, length or width depending on the geometry of the second wall.

Distal portion 36 is configured to visualize and manipulate tissue, for example, to dissect tissue and/or muscle, such as the psoas muscle adjacent a surgical site in the non-expanded configuration. Bifurcated cylinder 42 has a smooth or even inner and outer surface. It is envisioned that all or only a portion of the inner and outer surfaces of bifurcated cylinder 42 may have alternate surface configurations, such as, for example, rough, threaded for connection with other instruments, arcuate, undulating, porous, semi-porous, dimpled, polished and/or textured according to the requirements of a particular application. It is envisioned that distal portion 36 and/or distal cavity 44 may have alternate cross section configurations, such as, for example, oval, oblong, triangular, rectangular, square, polygonal, irregular, uniform, non-uniform, variable and/or tapered. It is further contemplated that distal portion 36 may include fastening elements such as anchors, detents and/or openings for connection to surgical instruments.

Distal portion 36 has a length extending from proximal portion 34, which extends to a surgical target. It is contemplated that distal portion 36 extends a length to avoid proximal portion 34 entering a body cavity of a patient an undesirable depth. In one embodiment, distal portion 36 extends from at least an incision to a surgical target in a configuration to prevent proximal portion 34 from entering the psoas tissue. In one embodiment, distal portion 36 extends from at least an incision to a surgical target. The distal end of distal portion 36 includes a tip 51 having a distal opening configured for direct visualization of a surgical site. In the non-expanded configuration, distal portion 36 and tip 51 are configured to dissect tissue and/or muscle to facilitate direct visualization of a surgical site with proximal portion 34, as will be described. In the expanded configuration, distal portion 36 and tip 51 is expanded from the non-expanded configuration, as described below, to facilitate passage of the surgical instrument therethrough. It is envisioned that tip 51 may have an alternative configuration relative to distal portion 36. It is envisioned that tip 51 may be pointed, planar or beveled. In one embodiment, distal portion 36 is approximately 4 centimeters (cm) in length,

Dilator 32 has a tapered transition 39 disposed between and for connecting proximal portion 34 with distal portion 36. Tapered transition 39 has a proximal end adjacent cylinder 38 having diameter d, and a distal end adjacent cylinder 42 having diameter d1. Diameter d1 of is less than diameter d such that tapered transition 39 is tapered from the proximal end to the distal end. The amount of taper may be adjusted depending upon, for example, the size and shape of the instrument supported in proximal cavity 40.

In the non-expanded configuration, transition 39 is configured to dissect tissue and/or muscle to facilitate direct visualization of a surgical site with proximal portion 34 and distal portion 36, as will be described. In the expanded configuration, transition 39 is expanded from the non-expanded configuration, as described below, to facilitate passage of the surgical instrument therethrough. Transition 39 has a smooth or even inner and outer surface. It is envisioned that all or only a portion of the inner and outer surfaces of transition 39 may have alternate surface configurations, such as, for example, rough, threaded for connection with other instruments, arcuate, undulating, porous, semi-porous, dimpled, polished and/or textured according to the requirements of a particular application. It is envisioned that transition 39 may have alternate cross section configurations, such as, for example, oval, oblong, triangular, rectangular, square, polygonal, irregular, uniform, non-uniform and/or variable. It is further contemplated that transition 39 may include fastening elements such as anchors, detents and/or openings for connecting proximal portion 34 to distal portion 36.

Dilator 32 includes a first arm 48 and a second arm 50. First arm 48 defines a portion of proximal portion 34, a portion of transition 39 and a portion of distal portion 36. Second arm 50 defines a portion of proximal portion 34, a portion of transition 39 and a portion of distal portion 36. First arm 48 and second arm 50 are pivotally connected at a pivot point 56 such that first arm 48 is movable relative to second arm 50 about a common axis defined by pivot point 56 to move dilator 32 between the non-expanded configuration and the expanded configuration. In the non-expanded configuration, arm 48 is disposed in a flush engagement with arm 50. From the non-expanded configuration, arm 48 is pivoted about pivot point 56 relative to arm 50 to space apart the respective sections of cylinders 38, 42 to radially expand cavities 40, 44 to the expanded configuration.

Arms 48, 50 include arm extensions 54 of distal portion 36, which extend longitudinally from transition 39. Arm extensions 54 are disposed in parallel relation to define distal cavity 44 extending along longitudinal axis a. In the non-expanded configuration, arm extension 54 corresponding to arm 48 is disposed in a flush engagement with arm extension 54 corresponding to arm 50. From the non-expanded configuration, arm extension 54 corresponding to arm 48 is pivoted relative to area extension 54 corresponding to arm 50 to space apart the sections of cylinder 42 to radially expand cavity 44 to the expanded configuration.

Proximal portion 34 of dilator 32 is connected to a handle 52 such that proximal portion 34 and distal portion 36 are rotatable relative to handle 52 to move dilator 32 between the non-expanded configuration and the expanded configuration. Handle 52 is disposed adjacent proximal portion 34 and is configured for manipulation by a medical practitioner during use. Handle 52 includes opposing arms 53, which are spaced apart when dilator 32 is in the non-expanded position and converge to expand proximal cavity 40 and distal cavity 44 when dilator 32 is in the expanded position. Opposing arms 53 include a gripping surface configured for manipulation by a medical practitioner. It is contemplated that opposing arms 53 may be connected together at a pivot point 56 such that opposing arms 53 can be moved relative to one another about a common axis defined by pivot point 56. It is further contemplated that arms 53 may be disposed in a parallel orientation to effect axial translation of proximal portion 34 for adjusting position thereof.

Opposing arms 53 includes a locking member 58 movably connected to an arm 53 for locking engagement with a gear tooth rack 59 extending from an opposing arm 53. Locking member 58 includes a pawl that engages the teeth of rack 59 to fix arms 48, 50 in a particular orientation, such as, for example, supporting a surgical instrument, manipulating tissue and/or fixing the components of the surgical system at a surgical site. Upon disposal of dilator 32 in a desired orientation between and including the non-expanded configuration and the expanded configuration, the pawl of locking member 58 is manipulated to engage rack 59 to dispose dilator 32 in a locked orientation.

Dilator 32 includes a spring member 60 extending between and biasing opposing arms 53. Spring member 60 facilitates inadvertent movement of opposing arms 53 and is configured to provide a tactile ergonomic configuration and gripping surface to handle 52.

In the first position, dilator 32 is in a non-expanded configuration, as shown in FIGS. 1-3. In the first position, distal cavity 44 is configured to provide direct visualization of the surgical site and proximal cavity 40 is configured to support a surgical instrument. Dilator 32 may be inserted through an incision over a surgical site such that a surgeon may view the surgical site through proximal cavity 40 and distal cavity 44. From the first position, arm 48 is pivoted about pivot point 56 relative to arm 50 to space apart the respective sections of cylinders 38, 42 to radially expand cavities 40, 44 to the expanded configuration.

In the second position, dilator 32 is disposed in an expanded configuration, as shown in FIG. 4. In the second position, distal cavity 44 is expanded to a configuration such that the surgical instrument is movable along the longitudinal axis a. In particular, moving dilator 32 from the first position to the second position expands distal cavity 44 such that an instrument positioned within proximal cavity 40 moves longitudinally into and through distal cavity 44 to a surgical site along longitudinal axis a. As shown in FIG. 2, a surgical instrument, such as, for example, a retractor 62 is positioned within proximal cavity 40 of dilator 32 when dilator 32 is in the first position. It is envisioned that proximal portion 34 may include at least one channel extending through proximal portion 34 configured to facilitate passage of one or more blades of retractor 62.

In assembly, operation and use, surgical system 30, similar to that described above, is employed, for example, with a surgical procedure on a patient for a discectomy and/or fusion procedure. It is envisioned that surgical system 30 may be used in any existing surgical method or technique including open surgery, mini-open surgery and minimally invasive surgery including percutaneous. The components of surgical system 30 can be delivered or implanted as a pre-assembled device or can be assembled during a treatment. The components of surgical system 30 may be completely or partially revised, removed or replaced during a treatment.

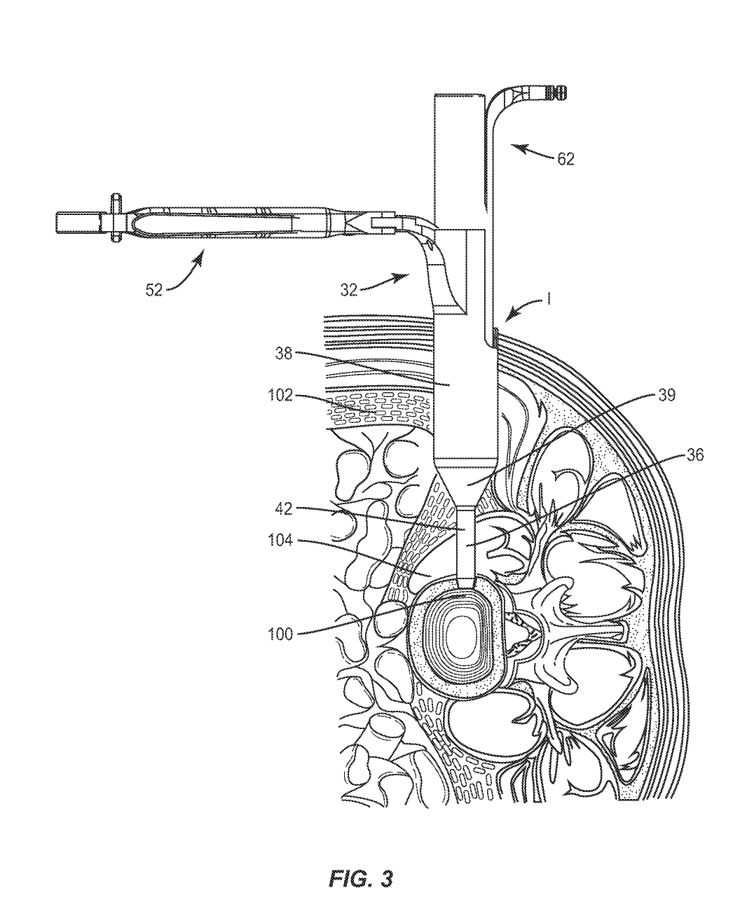

To gain access to a targeted surgical site, such as tissue and body cavities disposed adjacent a spine 100 through psoas tissue 104, as shown in FIG. 3, a surgeon employs a minimally invasive percutaneous technique and makes an incision I in the skin of a patient over and in approximate alignment with the surgical site. Dissection through a plurality of abdominal muscle layers allows the surgeon to access a retroperitoneal cavity 102. The surgeon may verify that he or she has reached retroperitoneal cavity 102 either visually or with finger palpation or other landmarks.

Upon reaching retroperitoneal cavity 102, a preparation instrument(s) may be inserted within retroperitoneal cavity 102 to sweep the peritoneal contents anterior and protect retroperitoneal cavity 102 as instruments are passed in and out of incision I. For example, a preparation instrument may be disposed through the incision and into retroperitoneal cavity 102. It is envisioned that the preparation instrument(s) may include a Cobb elevator, a surgical drill, a sleeved burr, rasps, curettes and/or a rotating tissue remover such as a rapid disc removal system that can be low profile to cut and remove disc and/or bone material simultaneously. The preparation instrument(s) is employed to remove tissue and fluids adjacent tissues and/or bone, scrape and/or remove tissue from vertebral surfaces, as well as aspirate and irrigate the region according to the requirements of a particular surgical application. The preparation instrument is removed from the incision thereafter.

A practitioner inserts dilator 32 through incision I while dilator 32 is in the first, non-expanded configuration. The practitioner accesses spine 100 laterally by inserting distal portion 36 of dilator 32 through retroperitoneal cavity 102 to psoas tissue 104. After dilator 32 is disposed for engagement with psoas tissue 104, dilator 32 may be docked on top of psoas tissue 104 and/or fixed to a hospital bed, or other object, to maintain dilator 32 in place. Distal portion 36 is advanced through psoas tissue 104 to the surgical site. Dilator 32 is employed to separate muscles and/or tissues to create a passageway along a desired trajectory to the surgical site through which the surgery may be performed.

Psoas tissue 104 may then be dissected with dilator 32 between and including the non-expanded and expanded configurations to provide access to the surgical site. Dilator 32, via handle 52, may be manipulated in an open and closing motion to dissect and dilate tissue. Direct visualization of the tissue and/or muscle to be dissected is provided through proximal portion 34, transition 39 and distal portion 36. It is envisioned that the surgical instrument, such as, for example, retractor 62 defines a cavity that provides direct visualization of the surgical and/or the tissue to be manipulated while being supported by proximal portion 34.

Upon desired positioning of dilator 32 adjacent the surgical site, dilator 32 may be moved from the first, non-expanded configuration to the second, expanded configuration, as described above. From the first configuration, bifurcated cylinder 38 and bifurcated cylinder 42 expand radially to expand proximal cavity 40 and distal cavity 44. This configuration allows retractor 62 to slide and/or be manipulated via engagement for movement through cavities 40, 44 to adjacent the targeted surgical site. A stability pin 63 may be inserted through retractor 62 and into tissue, such as, bone to fix surgical system 30 with the targeted surgical site. Retractor 62 maintains access to the targeted surgical site for a particular surgical treatment. Dilator 32 may be removed from the patient through incision I.

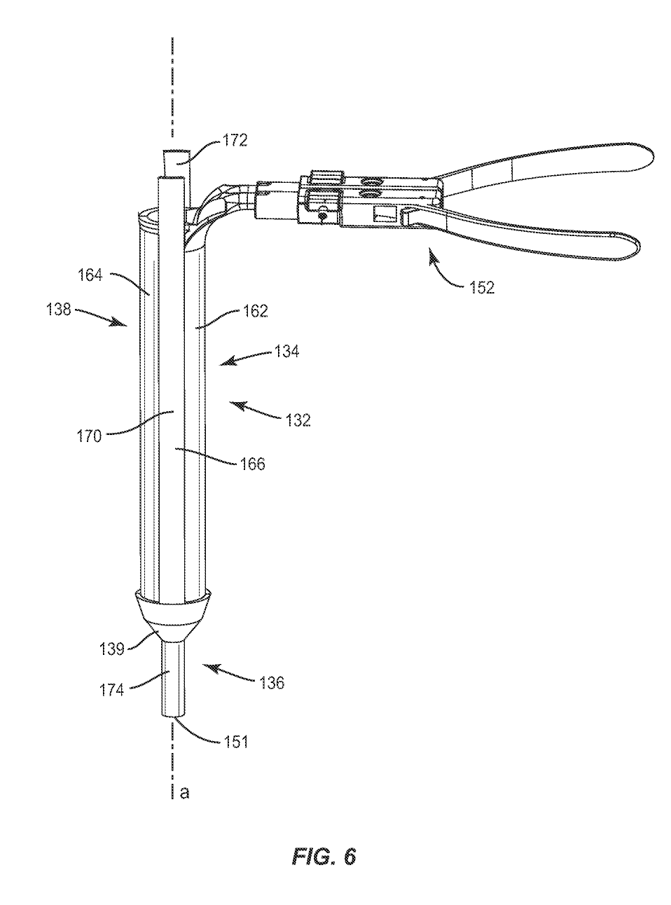

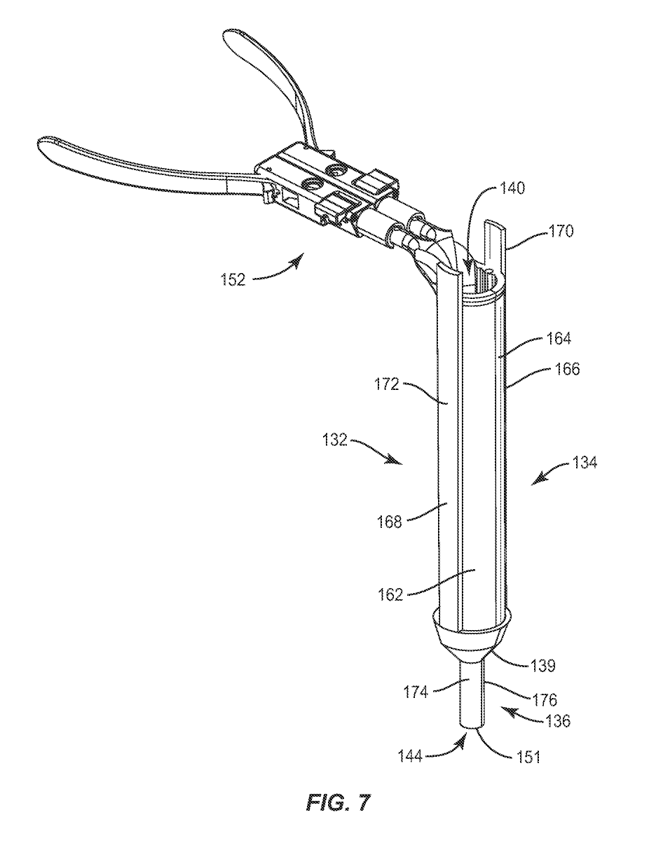

In one embodiment, as shown in FIGS. 6 and 7, system 30 includes a dilator 132, similar to dilator 32 described above, which includes a pair of retractor blades 162, 164 and a pair of removable dilator tips 166, 168 configured to provide lateral access to the spine through the psoas muscle to create a void between muscle fibers by using direct visualization to dissect through the psoas muscle and other tissue. Retractor blades 162, 164 and tips 166, 168 define a proximal portion 134 and a distal portion 136 of dilator 132. Distal portion 136 extends from proximal portion 134.

Dilator 132 is movable between a non-expanded configuration, similar to that shown in FIGS. 1-3, and an expanded configuration, similar to that shown in FIG. 4. It is contemplated that system 30 may include one or a plurality of removable dilator tips. Dilator 132 is configured to be inserted via lateral access into an incision over a surgical site to provide direct visualization of the surgical site and/or position a surgical instilment within the surgical site.

Retractor blades 162, 164 include a first wall, such as, for example a bifurcated cylinder 138 of proximal portion 1134. Bifurcated cylinder 138 defines a proximal cavity 140 extending along longitudinal axis a defined by dilator 132. Tips 166, 168 include proximal extensions 170, 172 of proximal portion 134 that engage an outer surface of blades 162, 164, respectively, to support blades 162, 164 during use of dilator 132. Proximal extensions 170, 172 define a first cross section width.

Tips 166, 168 include distal extensions 174, 176 of distal portion 136, which extend longitudinally from a transition 139 disposed between and for connecting proximal portion 134 with distal portion 136. Distal extensions 174, 176 define a second cross section width, which is less than the first cross section width such that tapered transition 139 is tapered from the proximal end to the distal end. Distal extensions 174, 176 are disposed in parallel relation to define distal cavity 144 extending along longitudinal axis a. Distal extensions 174, 176 are configured to manipulate tissue, similar to that described above with regard to FIGS. 1-5. The distal end of distal extensions 174, 176 define a tip 151 having a distal opening configured for direct visualization of a surgical site. Proximal portion 134 of dilator 132 is connected to a handle 152, similar to handle 52 described above with regard to FIGS. 1-5.

In a first position, dilator 132 is disposed in a non-expanded configuration. In the first position, proximal cavity 140 and distal cavity 144 provide direct visualization of the surgical site and proximal extensions 170, 172 support retractor blades 162, 164. Dilator 132 may be inserted through an incision over a surgical site such that a surgeon may view the surgical site through proximal cavity 140 and distal cavity 144. In the second position, dilator 132 is disposed in an expanded configuration. In the second position, proximal cavity 140 and distal cavity 144 can be expanded.

In assembly, operation and use, surgical system 30, similar to that described above, is employed, for example, with a surgical procedure on a patient for a discectomy and/or fusion procedure. A practitioner inserts dilator 132 through an incision while dilator 132 is in the first, non-expanded configuration. Dilator 132 is docked on top of psoas tissue to maintain dilator 132 in place. Distal portion 136 is advanced through psoas tissue to the surgical site. Dilator 132 is employed to separate muscles and/or tissues to create a passageway along a desired trajectory to the surgical site through which the surgery may be performed, similar to system 30 described above.

Psoas tissue is dissected with dilator 132 between and including the non-expanded and expanded configurations to provide access to the surgical site. Dilator 132, via handle 152, may be manipulated in an open and closing motion to dissect and dilate tissue. Direct visualization of the tissue and/or muscle to be dissected is provided through proximal portion 134, transition 139 and distal portion 136.

Upon desired positioning of dilator 132 adjacent the surgical site, dilator 132 may be moved from the first, non-expanded configuration to the second, expanded configuration, as described above. From the first configuration, proximal portion 134, transition 139 and distal portion 136 expand radially to expand proximal cavity 140 and distal cavity 144. Tips 166, 168 are removed from retractor blades 162, 164 and the surgical site. A stability pin may be inserted through retractor blades 162, 164 and into tissue to fix surgical system 30 with the targeted surgical site. Retractor blades 162, 164 are disposed to remain with the surgical site to maintain access to the surgical site for a particular surgical treatment.

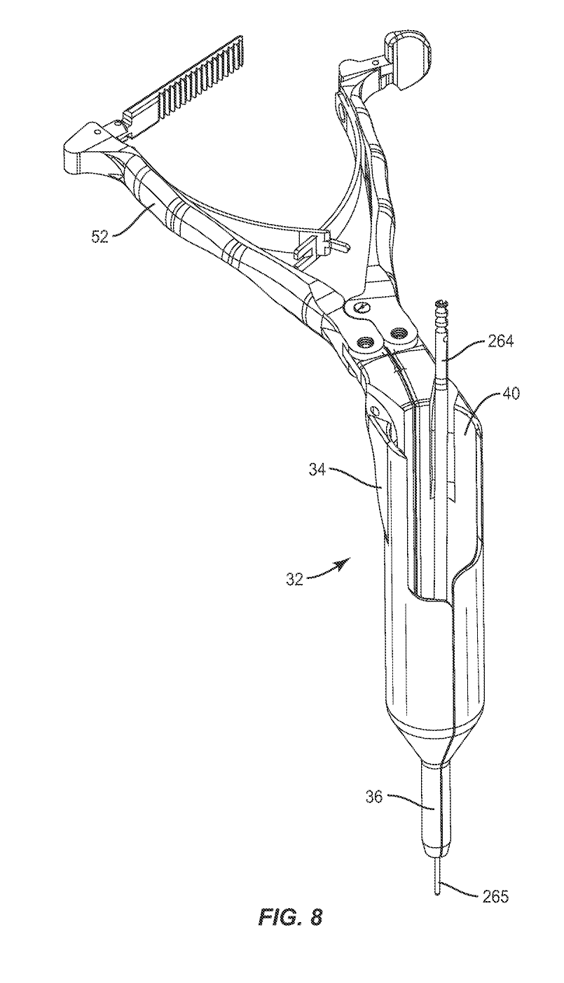

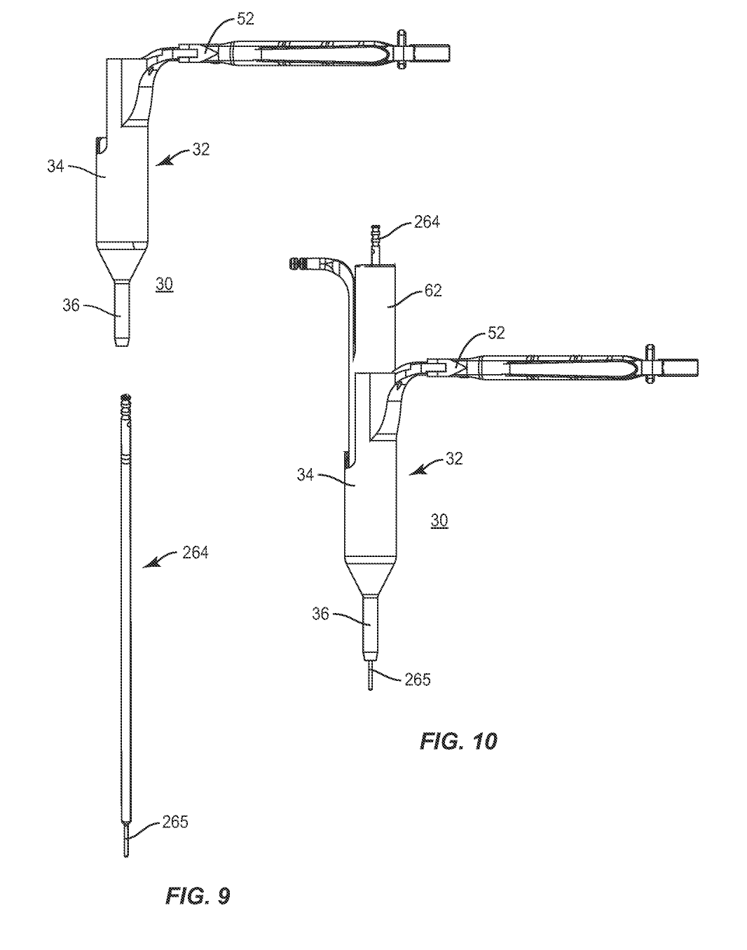

in one embodiment, as shown in FIGS. 8-11, system 30 described above with regard to FIGS. 1-5 includes a stimulated probe 264. Stimulated probe 264 is configured for passage through psoas tissue to determine a passageway to a spine that avoids undesirable engagement or interference with body structures, for example, neural structures and/or tissue in a configuration and orientation to avoid surgical and post-surgical complications. It is contemplated that stimulated probe 264 may be equipped with one or more electrodes for use in detecting the existence of neural structures. As such, it is envisioned that system 30 can be employed to determine the proximity of neural elements. Stimulated probe 264 is operable to deliver an electrical signal to a location in the patient's body to monitor proximity of neural elements adjacent a distal end 265 of stimulated probe 264. A lead connects stimulated probe 264 to an electrical signal source (not shown), which may comprise a portion of a nerve monitoring system. See, for example, the NIM-Spine™ System marketed by Medtronic, Inc. or any other suitable nerve monitoring system.

Stimulated probe 264 is configured to detect the existence, distance and/or direction of neural structures during the distraction, retraction and dissection of tissue by dilator 32. Stimulated probe 264 detects the presence of nerves through the application of a stimulation signal and monitoring the evoked signals associated with the nerves disposed adjacent the passageway and/or operative corridor being created by system 30, in accordance with the principles of the present disclosure.

In assembly, operation and use, surgical system 30, similar to that described above, is employed, for example, with a surgical procedure on a patient for a discectomy and/or fusion procedure. Stimulated probe 264 is inserted through an incision and passed through psoas tissue to determine a safe passageway to a spine. It is contemplated that stimulated probe 264 may also provide dilation.

A practitioner inserts dilator 32 through an incision while dilator 32 is in the first, non-expanded configuration over stimulated probe 264. Dilator 32 is docked on top of psoas tissue to maintain dilator 32 in place. Distal portion 36 is advanced through psoas tissue to the surgical site. Retractor 62 is disposed with proximal portion 34, as described above. Dilator 32 is employed to separate muscles and/or tissues to create a passageway along a desired trajectory to the surgical site through which the surgery may be performed, as described above. Upon desired positioning of dilator 32 adjacent the surgical site, dilator 32 may be moved from the first, non-expanded configuration to the second, expanded configuration, as described above, and retractor 62 is manipulated via engagement for movement through cavities 40, 44 to adjacent the targeted surgical site. It is envisioned that stimulated probe 264 is replaced with a guidewire and/or small dilator. Retractor 62 is disposed to remain with the surgical site to maintain access to the surgical site for a particular surgical treatment, as described above, and dilator 32 is removed from the surgical site.

In one embodiment, similar to that described above with regard to FIGS. 8-11, system 30 includes a stimulated probe having a ball tip configuration. The stimulated ball tip probe is delivered to a surgical site through a stability pin channel, then the psoas muscle, and passed down to bone. Upon reaching bone at the surgical site, a stability pin is inserted through a retractor blade and into the bone. It is envisioned that a longer than typical stability pin is employed, which facilitates passage through the psoas tissue due to the thickness of the psoas tissue. It is contemplated that the stability pin may be inserted at various stages of a surgical procedure. It is further contemplated that the distal end of a surgical instrument, such as a retractor may be passed through a surgical site and directly into engagement with bone.

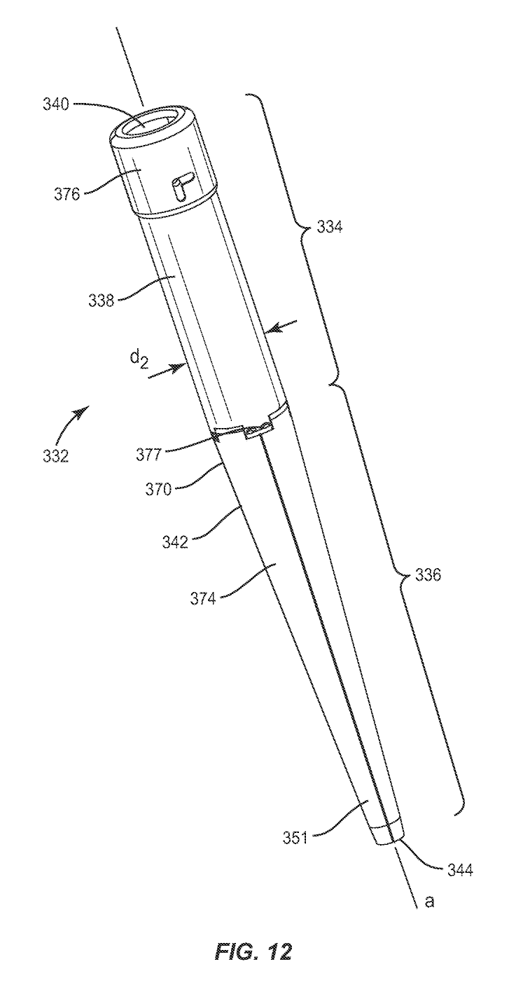

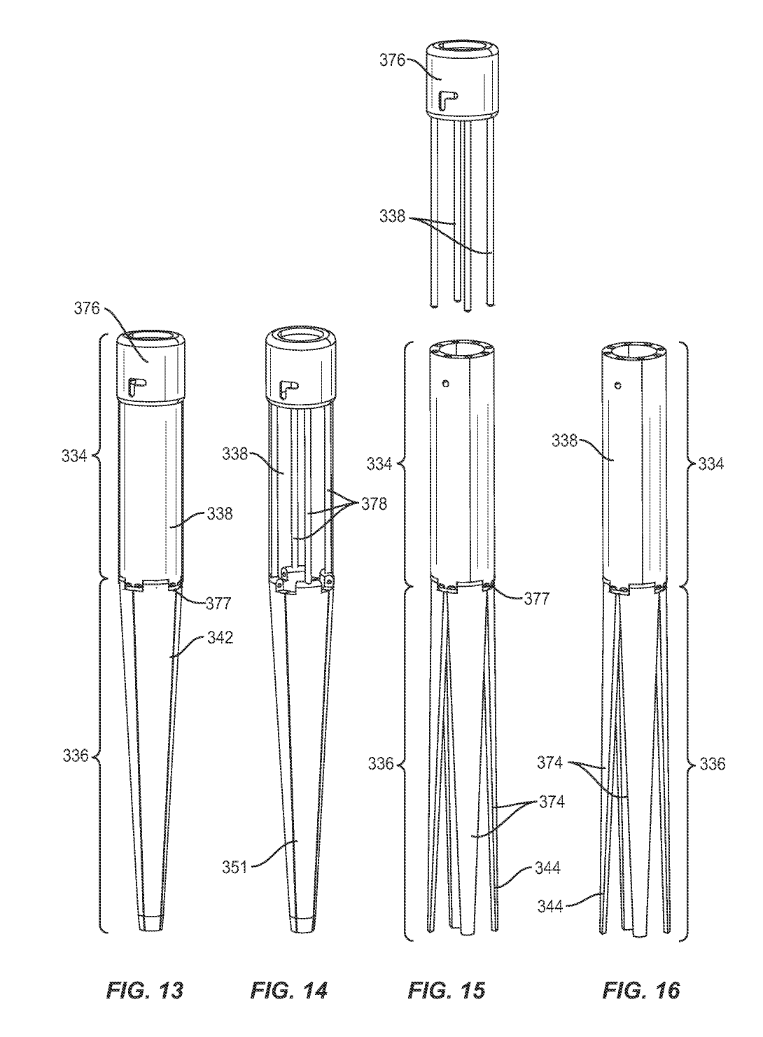

In one embodiment, shown in FIGS. 12-16, system 30 includes a dilator 332, similar to dilator 32 described above, which includes a proximal portion 334 and a tapered distal portion 336 extending from proximal portion 334. Dilator 332 is movable between a first, non-expanded configuration, as shown in FIG. 12, and a second, expanded configuration, as shown in FIG. 16. Dilator 332 is configured to be inserted via lateral access into an incision over a surgical site to provide direct visualization of the surgical site and/or position a surgical instrument within the surgical site.

Proximal portion 334 of dilator 332 includes a first wall, such as, for example a cylinder 338. Cylinder 38 defines a first cavity, such as, for example, a proximal cavity 340 extending along longitudinal axis a defined by dilator 332. Cylinder 338 has a first cross section dimension, such as, for example, a diameter d2 of proximal portion 334. Proximal portion 334 includes a cap 376 configured to be mounted with a proximal end of proximal portion 334. Cap 376 includes legs 378 that are slidably received within corresponding cavities of cylinder 338. Cap 376 is removable to accommodate and facilitate passage of a surgical instrument through proximal portion 334.

In the non-expanded configuration, proximal portion 334 is configured to support a surgical instrument within proximal cavity 340 and facilitate direct visualization of a surgical site with distal portion 336. In the expanded configuration, proximal portion 334 is expanded from the non-expanded configuration to facilitate passage of the surgical instrument therethrough. Distal portion 336 of dilator 332 includes a second wall, such as, for example, an expanding cone 342. Cone 342 is configured to define a distal cavity 344 extending along longitudinal axis a. Cone 342 has a second cross section dimension, such as, for example, a decreasing diameter that is less than diameter d2 of cylinder 338.

Distal portion 336 is configured to visualize and manipulate tissue, for example, to dissect tissue and/or muscle, such as the psoas muscle adjacent a surgical site in the non-expanded configuration. The distal end of distal portion 336 includes a tip 351 having a distal opening configured for direct visualization of a surgical site. In the non-expanded configuration, distal portion 336 and tip 351 are configured to dissect tissue and/or muscle to facilitate direct visualization of a surgical site with proximal portion 334. In the expanded configuration, distal portion 336 and tip 351 is expanded from the non-expanded configuration to facilitate passage of the surgical instrument therethrough. In one embodiment, distal portion 336 is approximately 4 centimeters (cm) in length.

Distal portion 336 includes hingedly connected extensions 374. Extensions 374 are rotatable about pivot connections 377 disposed adjacent a distal end of proximal portion 334. Extensions 374 are disposed in relation to define distal cavity 344 extending along longitudinal axis a. In the non-expanded configuration, extensions 374 are disposed in a flush engagement and tapered configuration to dissect tissue and/or muscle to facilitate direct visualization of a surgical site. From the non-expanded configuration, extensions 374 are pivoted radially outward relative to proximal portion 334 to radially expand cavity 344 to the expanded configuration. In one embodiment, the surgical instrument passes through proximal cavity 340 to engage extensions 374. This engagement causes extensions 374 to pivot radially outward.

In the first position, dilator 332 is in a non-expanded configuration, as shown in FIG. 12, In the first position, distal cavity 344 is configured to provide direct visualization of the surgical site and proximal cavity 340 is configured to support a surgical instrument. Dilator 332 may be inserted through an incision over a surgical site such that a surgeon may view the surgical site through proximal cavity 340 and distal cavity 344. From the first position, extensions 374 are pivoted radially outward relative to proximal portion 334 to radially expand cavity 344 to the expanded configuration.

In the second position, dilator 332 is disposed in an expanded configuration, as shown in FIG. 16. In the second position, distal cavity 344 is expanded to a configuration such that the surgical instrument is movable along longitudinal axis a. In particular, moving dilator 332 from the first position to the second position expands distal cavity 344 such that an instrument positioned within proximal cavity 340 moves longitudinally into and through distal cavity 344 to a surgical site along longitudinal axis a.

In assembly, operation and use, surgical system 30, similar to that described above and with regard to FIGS. 8-11, is employed, for example, with a surgical procedure on a patient for a discectomy and/or fusion procedure. A stimulated probe, similar to stimulated probe 264 described above, is inserted through an incision and passed through psoas tissue to determine a safe passageway to a spine.

A practitioner inserts dilator 332 through an incision while dilator 332 is in the first, non-expanded configuration over the stimulated probe. Distal portion 336 is advanced through psoas tissue to the surgical site. A surgical instrument, such as, for example, a retractor is disposed with proximal portion 334. Dilator 332 is employed to separate muscles and/or tissues to create a passageway along a desired trajectory to the surgical site through which the surgery may be performed, as described above. The retractor passes through proximal cavity 340 to engage extensions 374, causing extensions 374 to pivot radially outward such that dilator 332 is disposed in the expanded configuration, as described above. The retractor passes through dilator 332 to the surgical site and is disposed to remain with the surgical site to maintain access to the surgical site fir a particular surgical treatment, as described above, and dilator 332 is removed from the surgical site. It is contemplated that dilator 332 is hinged to be easily removable from underneath retractor arms. It is further contemplated that dilator 332 is made from multiple components that separate and slide out and away from the retractor once the retractor is in final position.

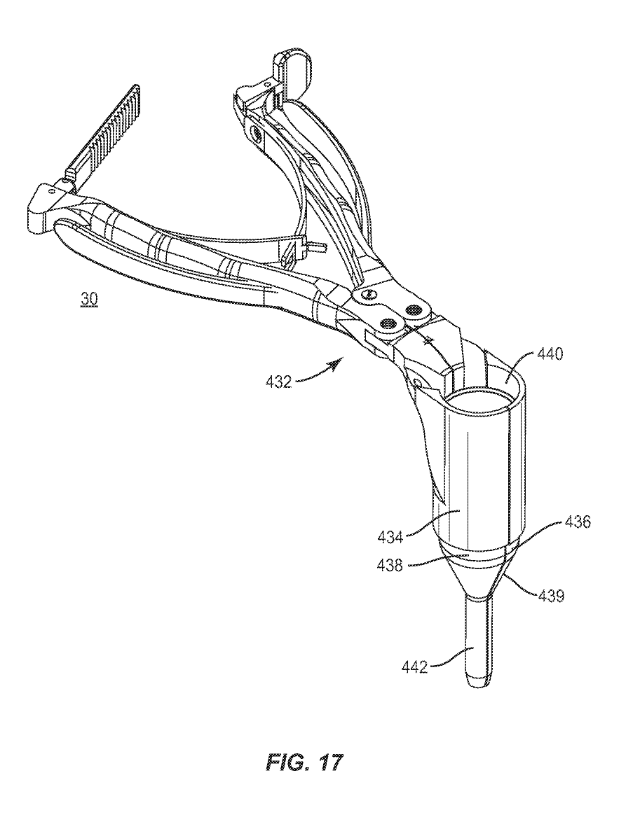

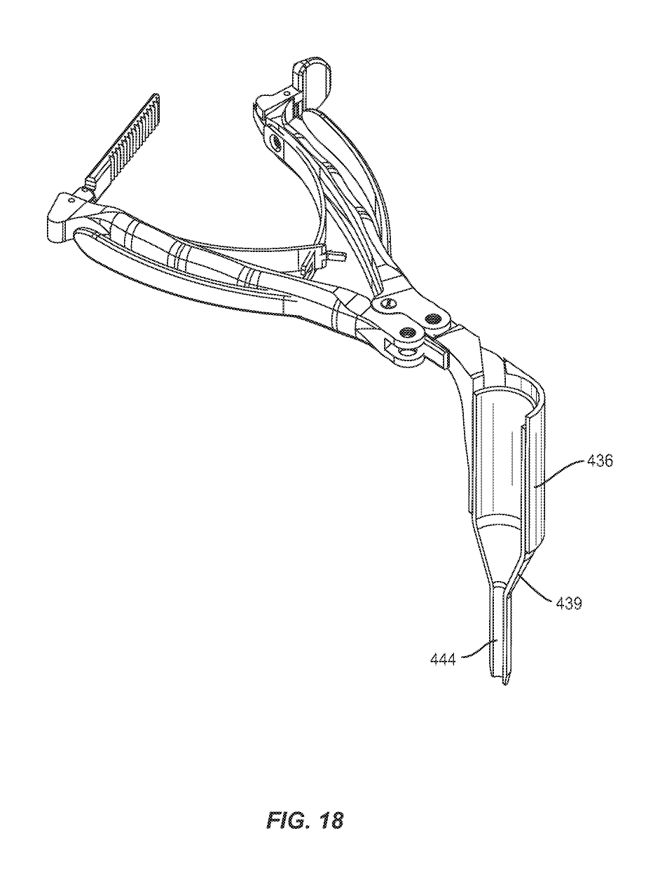

In one embodiment, as shown in FIGS. 17-20, system 30 includes a dilator 432, similar to dilator 32 described above, which includes a proximal portion having an adjustable axial length such that dilator 432 is adjustable to conform to an outer surface of the patient. It envisioned that the proximal portion is adjustable in length so that when dilator 432 is docked, the top of dilator 432 and/or surgical instruments supported therewith can be at approximately skin level. For example, in surgical applications requiring a shorter axial length to conform to an outer surface of the patient, as shown in FIGS. 17-18, dilator 432 includes a proximal portion and a distal portion extending from the proximal portion. Dilator 432 is movable between a non-expanded configuration and an expanded configuration, similar to that discussed, for insertion via lateral access into an incision over a surgical site to provide direct visualization of the surgical site and/or position a surgical instrument within the surgical site.

The proximal portion of dilator 432 includes a proximal portion of a pair of short dilator blades 434, 436 and a bifurcated cylinder 438. Blades 434, 436 are mounted to cylinder 438 and defines a proximal cavity 440. In the non-expanded configuration, blades 434, 436 and cylinder 438 are configured to facilitate direct visualization of a surgical site with the distal portion, similar to distal portion 36 described above. It is contemplated that blades 434, 436 and cylinder 438 can support a surgical instrument within proximal cavity 440. In the expanded configuration, blades 434, 436 and cylinder 438 are expanded to space apart blades 434, 436 from the non-expanded configuration, similar to proximal portion 34 described above with regard to FIGS. 1-5, to facilitate passage of a surgical instrument therethrough.

The distal portion of dilator 432 includes a distal portion of blades 434, 436, which includes a bifurcated cylinder 442 configured to define a distal cavity 444. Cylinder 442 has a diameter of the distal portion of dilator 432 that is less than the diameter cylinder 438. The distal portion of dilator 432 is configured to visualize and manipulate tissue, for example, to dissect tissue and/or muscle, such as the psoas muscle adjacent a surgical site in the non-expanded configuration. Dilator 432 has a tapered transition 439, similar to transition 39 described above.

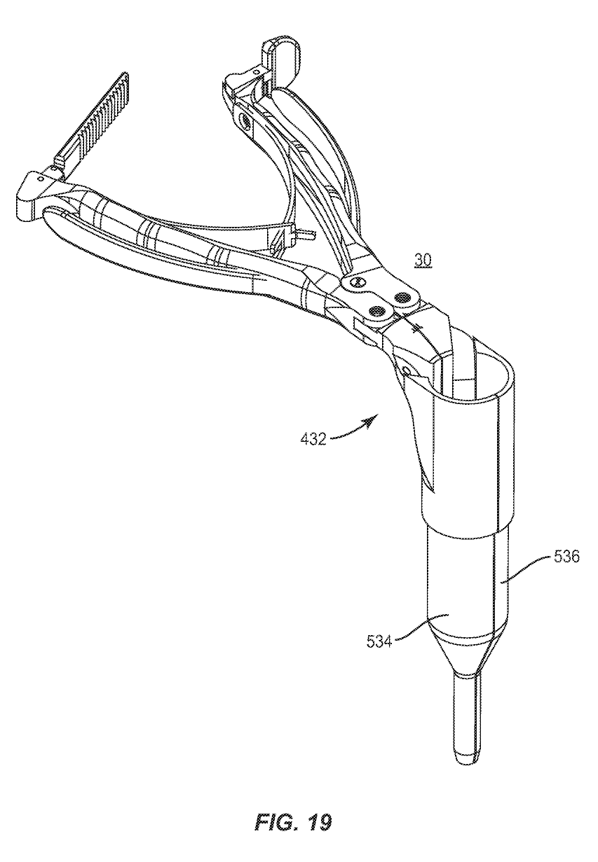

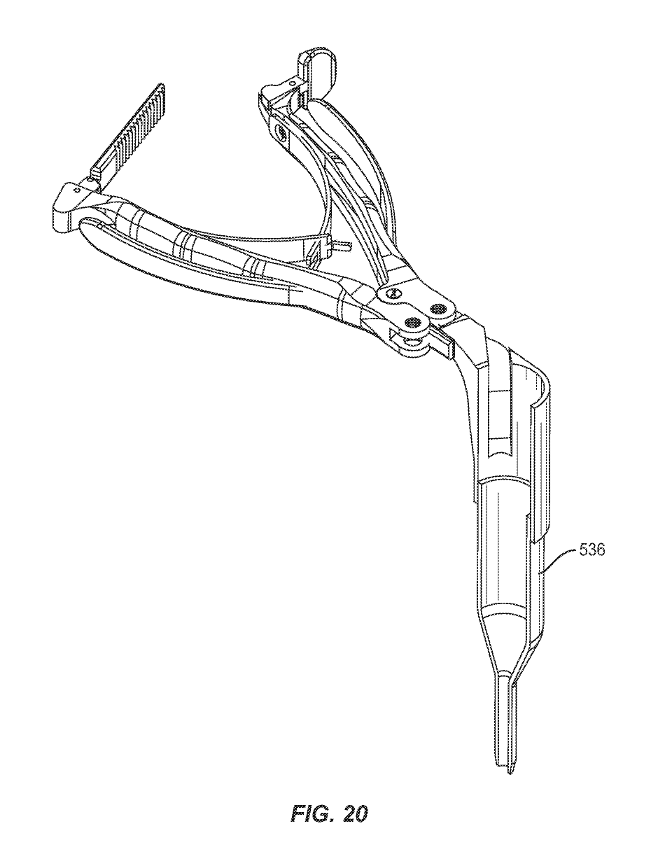

It is contemplated that that the axial length of the proximal portion of dilator 432 may be adjusted. For example, the surgical application may require a longer axial length than that provided by blades 434, 436. For adjustment of the axial length of dilator 432, a pair of long dilator blades 534, 536 are employed with dilator 432 described above. As such, the proximal portion of dilator 432 includes long dilator blades 534, 536, which extend a greater length to conform to an outer surface of the patient, as shown in FIGS. 19-20. Blades 534, 536, include the distal portion of dilator 432, a transition and are movable between a non-expanded and expanded configuration, similar to that described herein. It is envisioned that system 30 may include a plurality of dilator blades of various lengths for use with dilator 432.

System 30 may be employed for performing spinal surgeries, such as, for example, discectomy, laminectomy, fusion, laminotomy, laminectomy, nerve root retraction, foramenotomy, facetectomy, decompression, spinal nucleus or disc replacement and bone graft and implantable prosthetics including plates, rods, and bone engaging fasteners.

It will be understood that various modifications may be made to the embodiments disclosed herein. Therefore, the above description should riot be construed as limiting, but merely as exemplification of the various embodiments. Those skilled in the art will envision other modifications within the scope and spirit of the claims appended hereto.

What is claimed is:

1. A system for treating a spine, the system comprising:
a dilator defining a longitudinal axis and including a first portion and a second portion extending from the first portion, the dilator being movable between a first position and a second position,
the first portion including a first wall configured to define a first cross section dimension of the first portion and a first cavity extending along the longitudinal axis,
the second portion including a second wall configured to define a second cross section dimension of the second portion and a second cavity extending along the longitudinal axis, the second cross section dimension being less than the first cross section dimension, the second portion further defining an outer surface configured to dilate and/or dissect tissue adjacent a surgical site,
wherein in first position, the second cavity is configured to provide direct visualization of the surgical site and the first cavity is configured to support a surgical instrument, and in the second position, the second cavity is expandable to a configuration such that the surgical instrument is movable along the longitudinal axis in the second cavity.

2. The system of claim 1, wherein in the first position, the second cavity is disposed in a non-expanded configuration to provide direct visualization of the surgical site.

3. The system of claim 1, wherein the dilator includes a first arm and a second arm, the first arm defining at least a portion of the first portion and at least a portion of the second portion, and the second arm defining at least a portion of the first portion and at least a portion of the second portion, the first arm being movable relative to the second arm to expand the second cavity.

4. The system of claim 1, wherein the first portion is connected to a handle such that at least a portion of the second portion is rotatable relative to the handle to expand the second cavity.

5. The system of claim 1, wherein the first portion has a tapered configuration for connection with the second portion.

6. The system of claim 1, wherein the second portion includes arm extensions that engage in the first position and are spaced apart to expand the second cavity in the second position.

7. The system of claim 1, wherein the dilator includes a first arm and a second arm, the first arm defining at least a portion of the first portion and at least a portion of the second portion, and the second arm defining at least a portion of the first portion and at least a portion of the second portion, the first and second arms being removably attached to an outer surface of the surgical instrument.

8. The system of claim 1, wherein the second wall is radially expandable to expand the second cavity.

9. The system of claim 1, wherein the second wall is pivotable relative to the first portion to expand the second cavity.

10. The system of claim 1, wherein the second cavity is configured for disposal of a stimulated probe.

11. A speculum comprising:
a first arm; and
a second arm connected to the first arm and being movable relative to the first arm,
the first arm and the second arm defining a proximal portion of the speculum, the proximal portion including a first wall of the first arm and a first wall of the second arm, the first wall of the first arm and the first wall of the second arm being configured to define a cross section dimension of the proximal portion and a proximal cavity extending along a longitudinal axis thereof,
the first arm and the second arm further defining a distal portion of the speculum, the distal portion including a second wall of the first arm and a second watt of the second arm, the second wall of the first arm and the second wall of the second arm being configured to define a second cross section dimension of the distal portion and a distal cavity extending along the longitudinal axis, the second cross section dimension being less than the first cross section dimension, the distal portion further defining an outer surface configured to dilate and/or dissect tissue adjacent a surgical site,
wherein in first position, the distal cavity is disposable in a non-expanded configuration to provide direct visualization of the surgical site and the proximal cavity is configured to support a surgical instrument, and in the second position, the distal cavity is expandable such that the second wall of the first arm and the second wall of the second arm are spaced apart and the surgical instrument is movable along the longitudinal axis in the distal cavity.

12. The speculum of claim 11, wherein the first arm and the second arm are connected to a handle such that at least a portion attic distal portion is rotatable relative to the handle to expand the distal cavity.

13. A method for providing surgical access to a spine, the method comprising the steps of:
providing a dilator including a first portion and a second portion extending from the first portion, the first portion including a first wall configured to define a first cross section dimension of the first portion and a first cavity, the second portion including a second wall configured to define a second cross section dimension of the second portion and a second cavity, the second cross section dimension being less than the first cross section dimension;
disposing the dilator for engagement with psoas tissue of a patient such that the second cavity is non-expanded and oriented to provide direct visualization of a surgical site, and the first cavity is configured to support a surgical instrument;
advancing the second portion through the psoas tissue to the surgical site;
expanding the second cavity to a configuration such that the surgical instrument is movable along the longitudinal axis in the second cavity; and
advancing the surgical instrument to the surgical site to provide access thereto.

14. The method for providing surgical access to a spine of claim 13, wherein the step of advancing the second portion through the psoas tissue to the surgical site includes dissecting and/or dilating the psoas tissue with an outer surface of the second portion while providing direct visualization of the surgical site through the second cavity.

15. The method for providing surgical access to a spine of claim 13, wherein the step of advancing the second portion through the psoas tissue to the surgical site includes manipulating the second portion between a non-expanded and expanded position to dissect and/or dilate the psoas tissue with an outer surface of the second portion.

16. The method for providing surgical access to a spine of claim 13, wherein the step of disposing the dilator for engagement with psoas tissue includes the first cavity being configured to support a surgical retractor movable along the longitudinal axis in the second cavity and being advanced to bone at the surgical site.

17. The method for providing surgical access to a spine of claim 13, further comprising the step of advancing a stimulated probe through the second cavity to identify a passageway through the psoas tissue,

18. The method for providing surgical access to a spine of claim 13, wherein the step of expanding the second cavity includes the second wall being radially expandable to expand the second cavity.

19. The method for providing surgical access to a spine of claim 13, wherein the step of expanding the second cavity includes the second wall being pivotable relative to the first portion to expand the second cavity.

20. The method for providing surgical access to a spine of claim 13, wherein the step of providing a dilator includes a first portion having an adjustable axial length such that the dilator is adjustable to conform to an outer surface of the patient.
